(19) United States
(12) Patent Application Publication
(10) Pub. No.: US 2026/0290601 A1
(43) Pub. Date: Sep. 24, 2026

(54) METHOD FOR CONTINUOUSLY PREDICTING AN EVENT OF A PATIENT

(71) Applicants: B.G. Negev Technologies and Applications Ltd., Beer-Sheva (IL); The Institute of Computer Science of the Polish Academy of Sciences, Warsaw (PL)

(72) Inventors: Robert MOSKOVITCH, Beer Sheva (IL); Nevo ITZHAK, Beer Sheva (IL); Szymon JAROSZEWICZ, Warsaw (PL)

(21) Appl. No.: 19/179,908

(22) Filed: Apr. 15, 2025

Related U.S. Application Data
(60) Provisional application No. 63/634,481, filed on Apr. 16, 2024.

(51) Int. Cl. G16H 50/30

(52) U.S. Cl. CPC G16H 50/30

(57) ABSTRACT

The invention relates to a method for real time predicting an event of interest of a specific patient, comprising: (A) an offline procedure comprising: (a) providing a retrospective patients' database; (b) defining an event of interest; (c) performing data abstraction on raw data of the retrospective database, resulting in a database of STIs (symbolic time intervals); (d) performing TIRP mining given a minimal vertical support on the database of STIs, resulting in a collection of frequent TIRPs (time intervals related patterns); (e) for the event of interest, selecting from the collection of frequent TIRPs those who end with the event of interest, or receiving from an external source one or more TIRPs that end with the event of interest; (f) based on said retrospective database, calculating a prediction model m for each TIRP ending with the event of interest, that indicates, for each prefix or a partial prefix of that TIRP, the probability of the prefix or partial prefix, respectively, if will appear in real time, to complete, and optionally when, with the occurrence of said event of interest;

and,

(B) a real-time TIRP-based event of interest prediction procedure comprising: (g) continuously abstracting at each given time newly received raw data relating to the specific patient, thereby creating an unfolding STI series; (h) For each given TIRP that ends with the event of interest, continuously detecting from the abstracted data a currently unfolding TIRP prefix; (i) given the currently unfolding prefix and a specific event of interest, referring to the model and receiving from the model m the respective probability of the unfolding prefix to complete at the event of interest; and (j) continuously displaying to a user the probability of the currently unfolding prefix to complete with the event of interest.

### CROSS-REFERENCE TO RELATED APPLICATIONS

This application claims priority to US 63/634,481 filed Apr. 16, 2024, the entire contents of which are hereby incorporated by reference.

### FIELD OF THE INVENTION

The invention generally relates to the field of medically monitoring a patient, for example, a patient within an Intensive Care Unit (ICU). More specifically, the invention relates to a method for predicting in real time whether a current state of a patient will develop to a specific event, and optionally when the event will occur.

### BACKGROUND OF THE INVENTION

In real-world applications, data is typically collected from multiple data sources, resulting in heterogeneous multivariate temporal data. This means that the data may be from different sources, with different types of variables, and with different sampling frequencies. For example, the data may include variables recorded at a fixed frequency (V^{FF}, such as temperature data that are sampled from the patient every 3 hours), obtained by irregular sampling (V^{IS}, such as blood tests that are only sampled when a certain event occurs), represented by instantaneous events (V^{IE} such as an alarm trigger e.g., ICU monitor beeping due to a threshold exceeded), or events with duration (V^{STIs}, such as a duration that a patient stays in the hospital).

In many real-life data science problems, in which data is gathered from various sources, the multivariate temporal data are heterogeneous. Some variables may be sampled regularly but at different frequencies (e.g. sensor measurements) and some variables irregularly (e.g. variables measured manually). Other temporal variables may be represented by events that may or may not have varying duration.

In hospitalized patient data, there are databases in which the start and end times of events are stored (or can be determined), resulting in time interval data. Discovering patterns from time interval data can reveal informative patterns, in which the time intervals are related by temporal relations, such as before, meets, or overlap. When multiple temporal variables are sampled in a variety of forms, duration, and frequencies, as well as irregular events that may or may not have a duration, time intervals patterns are a powerful way to discover temporal knowledge, since these temporal variables can be transformed into a uniform format of time intervals. Predicting the completion of a pattern can be used when the pattern ends with an event of interest, such as the recovery of a patient, an undesirable event, such as a medical complication, etc. In recent years, an increasing number of studies have been published on time intervals-related patterns (TIRPs), their discovery, and their use as features for classification. However, so far no study has predicted the completion of a TIRP related to a patient, namely, the occurrence of one of the events mentioned above. The main challenge in performing such a completion prediction in real time occurs when the time intervals are coinciding and not finished yet, which introduces uncertainty in the evolving temporal relations and, thus, the TIRP's unfolding process.

In medicine, for example, a continuous prediction of a clinical outcome of a monitored patient in an intensive care unit (ICU) is critical, where the clinical team needs to be warned of potential complications early enough to allow early intervention and, ideally, prevent complications. For example, an acute blood pressure increase is an undesirable event extracted from real ICU patients.

The idea of predicting temporal patterns' completion was applied with sequential patterns in several domains, such as complex activity recognition and recommendations. However, patient data are not homogeneous. In some domains, events may last for a certain period, in which the starting and ending times are known and stored in a database, resulting in symbolic time interval data. Discovering patterns while considering events' starting and ending times can reveal more informative patterns. For example, a pattern from time interval data may be that hospitalized patients with COVID-19 frequently start with symptoms of “fever” and “cough,” and a week or so later also begin experiencing shortness of breath, in which the symptoms have not ended until the ICU admission time.

Traditional time-series analysis methods are often not designed to handle heterogeneous longitudinal data, as they are based on the assumption that all the temporal variables are sampled at the same fixed frequency. Approaches based on item sets ignore the order between the items, while approaches based on sequence mining are limited to analyzing instantaneous events (V^{IE}), and cannot analyze events with duration (V^{STIs}). In previous studies, models based on time interval patterns or symbolic time points achieved better classification performance than deep neural networks on the raw temporal data. An explanation for that could be the neural networks' limitations as they require a large amount of data, and have trouble handling sparsity which occurs when handling heterogeneous multivariate temporal data with different granularity.

It is an object of the present invention to provide a a real-time method that, from a current patient's condition, predicts the likelihood and timing of a future clinical event of interest.

Another object of the invention is to provide a method for predicting in real-time an event of interest of a patient, given heterogeneous types of data acquired from various sources.

It is still another object of the invention to provide the above prediction in higher prediction performance compared to prior art methods.

Another object of the invention is to utilize discovered time interval-related patterns (TIRPs) as interpretable, clinically meaningful features for real-time continuous prediction.

It is still another object of the invention to enable early warnings and actionable alerts for clinicians by predicting undesirable events before their actual occurrence.

It is still another object of the invention to enable early indication of a patient's tendency towards recovery, enabling boosting the patient's positive progress.

Other objects and advantages of the invention will become apparent as the description proceeds.

### SUMMARY OF THE INVENTION

The invention relates to a method for real time predicting an event of interest of a specific patient, comprising: (A) an offline procedure comprising: (a) providing a retrospective patients' database; (b) defining an event of interest; (c) performing data abstraction on raw data of the retrospective database, resulting in a database of STIs (symbolic time intervals); (d) performing TIRP mining given a minimal vertical support on the database of STIs, resulting in a collection of frequent TIRPs (time intervals related patterns); (e) for the event of interest, selecting from the collection of frequent TIRPS those who end with the event of interest, or receiving from an external source one or more TIRPs that end with the event of interest; (f) based on said retrospective database, calculating a prediction model m for each TIRP ending with the event of interest, that indicates, for each prefix or a partial prefix of that TIRP, the probability of the prefix or partial prefix, respectively, if will appear in real time, to complete, and optionally when, with the occurrence of said event of interest; and,

(B) a real-time TIRP-based event of interest prediction procedure comprising: (g) continuously abstracting at each given time newly received raw data relating to the specific patient, thereby creating an unfolding STI series; (h) For each given TIRP that ends with the event of interest, continuously detecting from the abstracted data a currently unfolding TIRP prefix; (i) given the currently unfolding prefix and a specific event of interest, referring to the model and receiving from the model m the respective probability of the unfolding prefix to complete at the event of interest; and (j) continuously displaying to a user the probability of the currently unfolding prefix to complete with the event of interest.

In an embodiment of the invention, the one or more TIRPs are a plurality of TIRP instances, all ending with the same event of interest, and wherein a plurality of prediction models m, each relating to a different from said plurality of TIRP instances, are grouped within a global prediction model M.

In an embodiment of the invention, a plurality probabilities received, respectively for each TIRP instance, are aggregated before displaying a single aggregated probability to the user.

In an embodiment of the invention, the prefix probabilities within each said model m are listed only for time interval end points (tieps) of the respective TIRP.

In an embodiment of the invention, the prefix probabilities within each model m are calculated by an SCPM (Segmented Continuous Prediction Model).

In an embodiment of the invention, the prefix probabilities within each model m are provided in a continuous time manner.

In an embodiment of the invention, the continuous time manner prefix probabilities within each said model m are calculated by either FCPM (Fully Continuous Prediction Model) or CPML (Machine Learning-Based Continuous Prediction Model).

In an embodiment of the invention, the calculation of each prefix probability within each model m is based on the rate in which same prefixes within the database of STIs have been eventually developed to a complete with the event of interest.

In an embodiment of the invention, the external source is a physician, a scientist, literature, or or any framework built on large-language-model technology.

In an embodiment of the invention, the method further displays to the user one of: (a) when the event of interest is expected to occur; and (b) the instances on which the displayed probability is based.

### BRIEF DESCRIPTION OF THE DRAWINGS

In the drawings:

FIG. 1a describes, in a flow diagram form, the offline procedure;

FIG. 1b describes in more detail a process 500 for creating the prediction model m;

FIG. 2a illustrates in a flow diagram form a real-time process 200a, according to a first embodiment of the invention;

FIG. 2b illustrates in a flow diagram form a real-time process 200b, according to a second embodiment of the invention;

FIG. 3 also shows several types of heterogeneous multivariate temporal data, typical to patient measurement items;

FIG. 4, shows the seven Allen' temporal relations between a pair of STIs;

FIG. 5(i) shows an example for a TIRP representation. FIG. 5(ii) shows a TIRP temporal relations conjunction;

FIG. 6 illustrates how a TIRP Q's completion probability is estimated at any given time point;

FIG. 7(i) and FIG. 7(ii) show examples for making prefix decisions given “temporary equals” and “temporary finished-by” cases, respectively;

FIG. 8 shows an example of measurement items and TIRPs detected in three patients;

FIG. 9 shows all the “phase changes” (tieps) occurring within a pattern extracted from one patient (patient 8);

FIG. 10 shows an unveiled partial TIRP comprising elements A and B* used for predicting whether the TIRP will eventually complete;

FIG. 11 provides an example of an entity (e.g., a patient) and its detected prefix instances of three TIRPs Φ={Q₁, Q₂, Q₃} that end with the event of interest y;

FIG. 12 shows an example where the event occurrence probability at any time point is based on the observed STI series. The prediction decision is made when the completion probability is higher than a threshold, and a time delay t has been passed;

FIG. 13 presents mean AUROC results for weighted aggregation functions, averaging the results of different t values;

FIG. 14 presents mean AUROC results for different event prediction models; and

FIG. 15 presents mean AUROC results for each model and t.

### DETAILED DESCRIPTION OF PREFERRED EMBODIMENTS

The health state of a patient staying in an ICU (for example) may end in various events of interest, such as recovery, a specific complication, or, unfortunately, death. Many factors are present and registered during the patient's stay, such as medications given, blood tests or other measurements, body temperature, etc. All these factors are recorded in the form of heterogeneous multivariate temporal data. The inventors have found that the progression of a patient toward one or another event (event of interest) statistically follows through specific determinate patterns. While these patterns may vary in duration or time of appearance, they are still deterministic to a high degree.

In some domains, events may last for a certain period, in which the starting and ending times are known and stored in a database, resulting in symbolic time interval data. Discovering patterns while considering events' starting and ending times can reveal more informative patterns. For example, a pattern from time interval data may be that hospitalized patients with COVID-19 frequently start with symptoms of “fever” and “cough” a week or so later, the patients also begin experiencing shortness of breath, in which the symptoms do not end until the ICU admission time.

In previous studies, models based on time interval related patterns or as classification features achieved better classification performance than deep neural networks on operating on raw temporal data. An explanation for that could be the neural networks' limitations as they require a large amount of data, and sparsity occurs when handling heterogeneous multivariate temporal data with different granularity. The present invention utilizes time interval patterns in a novel manner that differs from approaches used in the prior art. The invention introduces a novel methodology for continuous event of interest prediction estimation. Specifically, the invention provides a method for continuously predicting, in real-time, an event of interest, consisting of a temporal pattern that ends with the event and was discovered offline in a database, or was given from an external source, the probability that the pattern will be completed, meaning that the event of interest will occur based on a current patient's state. The invention may be applied to monitoring a patient and for the purpose of clinical event prediction.

The process of the present invention includes an offline procedure (aka training/learning stage) and a run-time process (also referred to as real-time). The offline procedure is generally performed once on a retrospective database, determining one or more patterns (TIRPs), each ending with a respective event of interest. One or more patterns may be associated with the same event of interest, or several patterns may be associated, each with a different event of interest. Although this description concentrates mainly on a single event of interest (or discovering multiple temporal patterns that end in that event), the same procedure can be run concurrently for any additional patterns and events. The offline procedure continues in determining, for each state of the pattern, the probability of the pattern to be completed, if that pattern will evolve during run-time. In one embodiment, this offline probability determination considers only specific states within the pattern while ignoring durations. In another embodiment, this offline probability determination considers durations and provides the respective probability of completing for any time during the pattern. During the patient's stay in the ICU (for example), and given the predetermined pattern, the associated event of interest, and the precalculated probabilities, the run-time process is repeatedly performed to determine and indicate the probability that the patient's current state will progress towards the respective event of interest. More specifically, the run-time procedure continuously indicates the probability that the relevant pattern will be completed with the event of interest.

It should be noted that in one embodiment, the patterns (TIRPs) and associated events of interest are determined by the offline procedure using data mining techniques. In another embodiment, one or more of the patterns are supplied from an external source, such as a physician or data scientist, defining the pattern based on his knowledge and experience, perhaps even assisted by the literature, or any other source, such as CHATGPT. The offline procedure then determines, for each state of the pattern, the respective probability of the entire pattern to be completed.

The patient's data is typically collected from multiple sources, resulting in heterogeneous multivariate temporal data. For example, data from different sources may include different types of variables with varying durations and sampling frequencies. Among others, the offline and run-time procedures of the invention bring the heterogeneous multivariate temporal data into a simplified representation that can be reliably handled and analyzed.

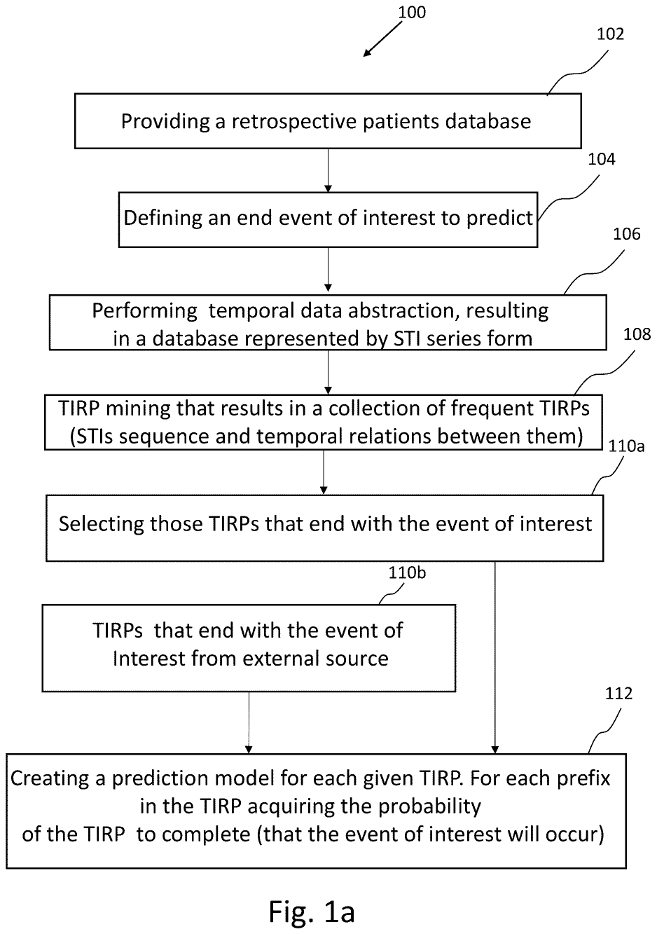

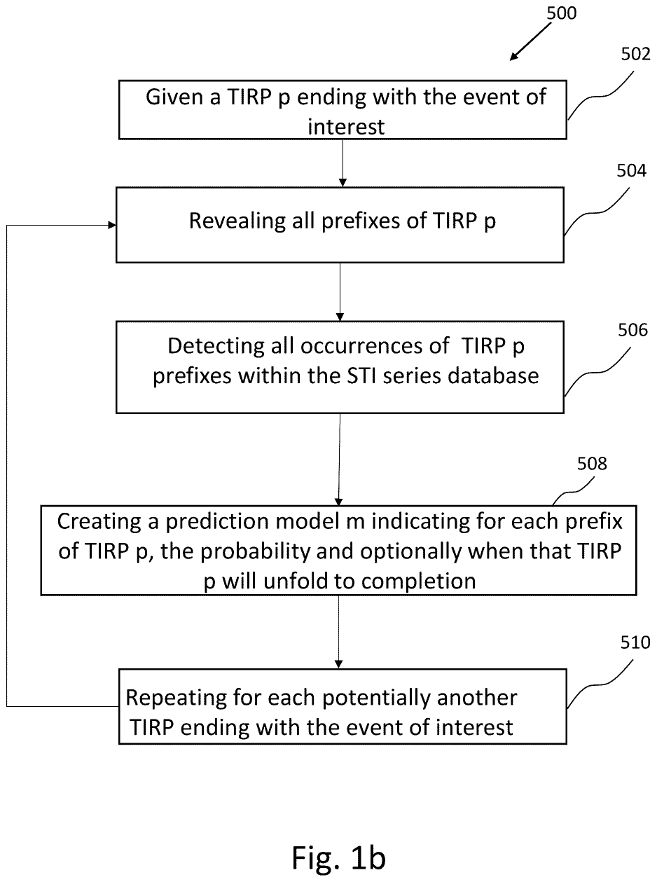

FIG. 1a describes, in a flow diagram form, the offline procedure. In step 102, a retrospective (for example, longitudinal) database containing retrospective patients records is provided. The retrospective database may (and preferably) be anonymous regarding specific patients' identities. In step 104, an event of interest is defined. An event of interest may be, for example, a patient recovery, one or more complications that occurred to patients, such as sepsis/acute hypertensive episodes, death of patients, etc. Next, a temporal data abstraction is performed on the entire database, resulting in a series of STIs for each patient. In the temporal data abstraction, sequences of measurements are concatenated based on their symbol, when they are the same and adjacent. For example, a sequence of body temperature measurements, all above a threshold (e.g., 37.0 degrees), may be grouped to form a single high (“H”) sequence. Similarly, a sequence of temperature measurements, all below the threshold of 37.0 degrees, may be grouped to form a single normal (“N”) sequence. Similarly, two thresholds may be defined to form three or more states (such as high “H”, medium “M”, and low “L”). The temporal data abstraction is performed only on specific types of measurements, as some other measurements are already in an STI form. The result of the temporal data abstraction of step 106 is an entire database represented uniformly by a series of STIs for each patient. In step 108, a TIRP mining (for example, using a vertical support) is performed on the database, resulting in a collection of frequent TIRPs (STIs and temporal relations between them). Various data mining algorithms may be used (as noted below, the inventors applied KarmaLego). In step 110a, those TIRPs ending with the event of interest are selected (one or more patterns may end in a same event of interest). Alternatively, or optionally, one or more TIRPs ending with the event of interest are provided from an external source, such as from a physician, a scientist, a literature source, or, or an LLM based frameworks such as CHATGPT. Finally, in step 112 a prediction model M (containing m1, m2, m3 . . . models for each distinct TIRP) is created for each given TIRP (selected in steps 110a or 110b). Each prediction model m contains, for each “prefix” (an exposed portion) of the TIRP, the probability that the TIRP will complete (meaning the respective event of interest will occur) and possibly when. As is elaborated hereinafter, each probability within prediction model m may be either discrete, i.e., includes calculated probabilities only at tieps of the STIs in the TIRP, for example, when the algorithm used is SCPM (Segmented Continuous Prediction Model which is not fully continuous), or continuous i.e., calculates probabilities also for times between tieps, for example, when one of FCPM (Fully Continuous Prediction Model), or CPML (Machine Learning-Based Continuous Prediction Model) algorithms is used. The SCPM, FCPM, and CPML algorithms are discussed in more detail below. A separate prediction model is created for each TIRP found in step 110a, or given in step 110b.

FIG. 1b describes in more detail a process 500 for creating the prediction model m. In step 502, a TIRP p ending with the event of interest is given. In step 504, all prefixes of TIRP p are revealed. In step 506, the procedure detects all occurrences of TIRP p prefixes appearing within the STI series database. In step 508, a prediction model m is created, indicating for each prefix (or partial prefix) of TIRP p, the probability and optionally when that TIRP p will unfold to completion. Finally, in step 510, if there are other TIRPs ending with the same event of interest, the procedure returns to step 504, and repeats steps 504, 506, and 508 for all additional TIRPS.

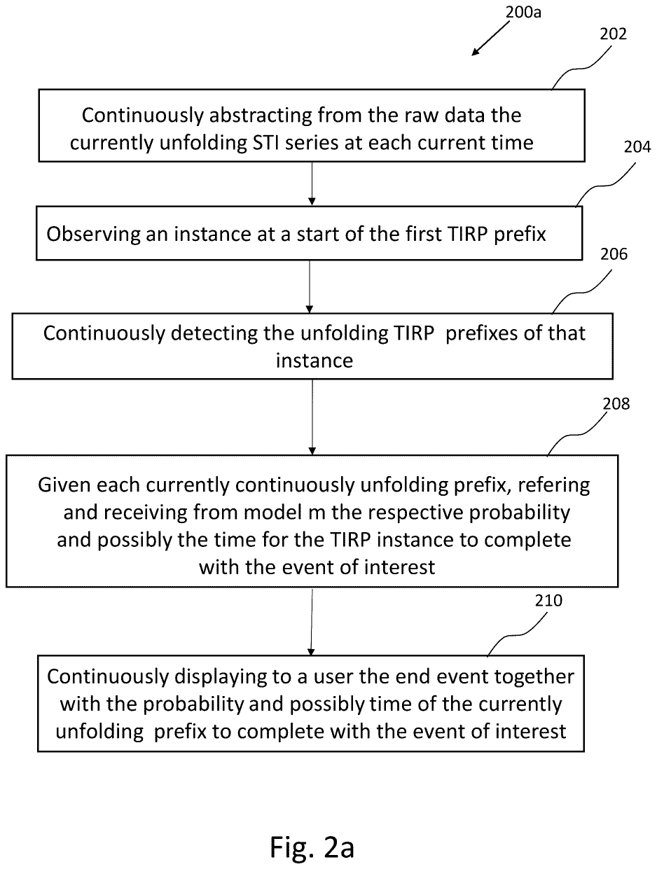

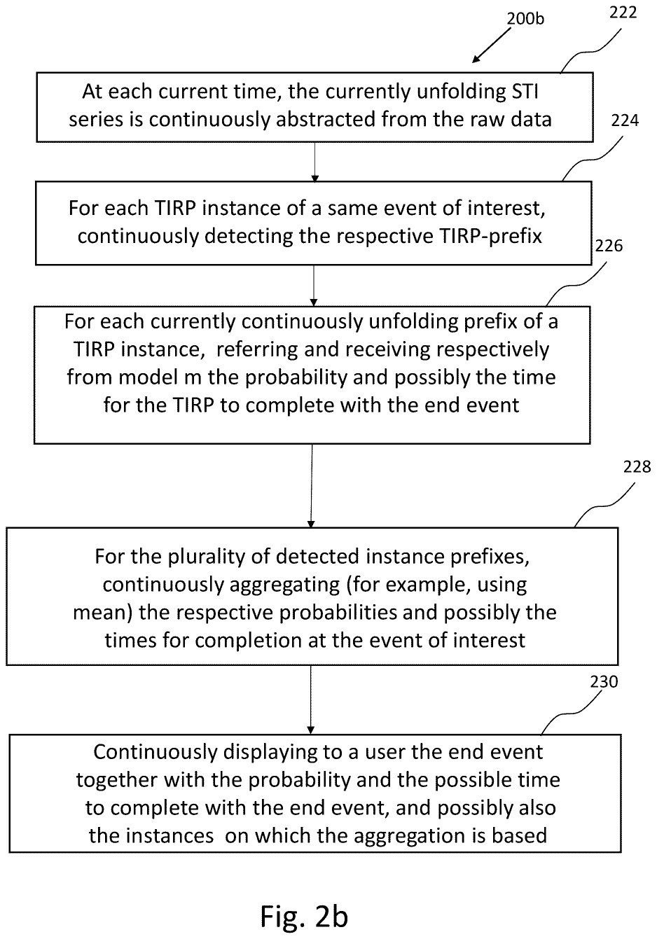

FIG. 2a illustrates in a flow diagram form a real-time process 200a, according to a first embodiment of the invention. For simplicity and generality, process 200a assumes that only one TIRP instance has been determined (or otherwise received) for the given end of interest. The real-time process 200a also assumes that the offline process 100 of FIG. 1a has been completed. Process 200b described later (FIG. 2b), refers to the case where two or more instances exist for the given event of interest. In step 202, and at each current time, the procedure continuously abstracts from the raw data the currently unfolding STI series. In step 204, an instance of a start of a first TIRP prefix is observed. In step 206, the procedure continuously detects the unfolding TIRP prefixes of that instance. Next, in step 208, and given each currently unfolding prefix, the procedure refers and receives from model m the respective probability, and possibly also the time, for the TIRP instance to complete with the event.

FIG. 2b illustrates in a flow diagram form a real-time process 200b, according to a second embodiment of the invention. Process 200b assumes that several TIRP instances have been determined (or otherwise received) for the given end of interest. The real-time process 200b also assumes that the offline process 100 of FIG. 1a has been completed. In step 222, the procedure continuously, and at each current time, abstracts from the raw data the currently unfolding STI series. In step 224, an instance of a start of a first TIRP prefix (or partial prefix) is observed. In step 226, and for each currently unfolding prefix of a TIRP instance, the procedure refers and receives respectively from model m the probability and possibly the time for the TIRP instance to complete with the event of interest. In step 228, and for the plurality of detected instance prefixes, the procedure continuously aggregates (for example, using mean) the respective probabilities and possibly the times for completion at the event of interest. Finally, in step 230 the procedure continuously displays to a user the event of interest together with the probability and the possible time to complete with the event of interest, and possibly also the instances on which the aggregation is based.

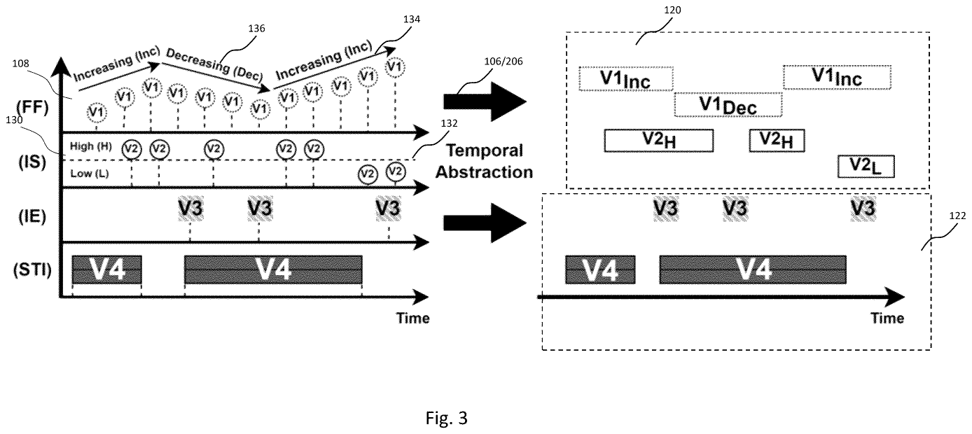

FIG. 3 illustrates various types of measurement items that are acquired from a patient, their abstraction according to the invention, and their conversion to STIs.

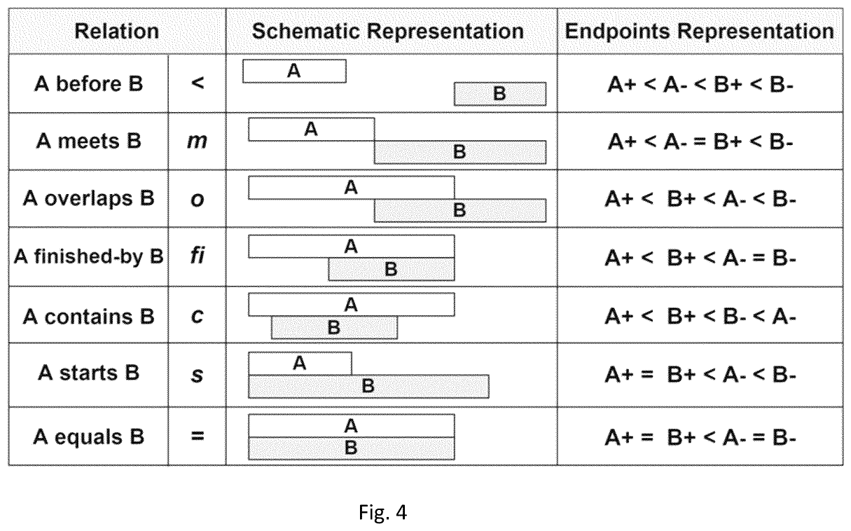

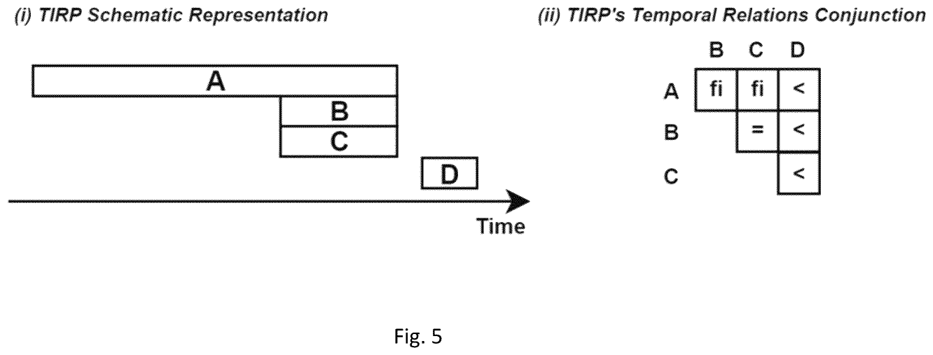

By its definition, a symbolic time interval (STI) includes a starting time point, an ending time point and a symbol from an ordered alphabet. For simplicity, the notation of an STI is identical to its symbol throughout the description. Additionally, A+ and A− are used when the starting and ending point of STI A are referred to (formal and full definitions provided hereinafter). Allen's temporal intervals algebra is used, as shown in FIG. 4, to represent the temporal relation between each pair of STIs, while other types of temporal relations can be used as well, such as their subsets, or disjunctions of few of them, or just other temporal relations. A time intervals-related pattern (TIRP) is defined by a set of ordered STIs and the conjunction of all the temporal relations between each pair of those STIs. For example, the TIRP illustrated in FIG. 5 is defined by four STIs (A, B, C, and D-FIG. 5(i)) and six temporal relations (FIG. 5(ii)). The durations of the TIRP STIs are not a part of their definition, which enables the discovery and detection of frequent TIRPs. Since the TIRP's STIs' duration may vary from different pattern's instances, the schematic representation (FIG. 5(i)) presents a possible instance of the pattern. The task of continuous estimation of a TIRP's completion is challenging, mainly due to the variability in the STIs' duration and gaps between the STIs of the pattern's instances, and the present invention provides a solution to this problem. The temporal variables of a monitored patient are heterogeneous, for example, due to lab test results, drug exposure periods, or the body's vital signs. As becomes apparent by the description, the time interval mining methods become suitable by the invention for heterogeneous multivariate data analysis given the transformation into a uniform representation of STIs, and the abstraction.

FIG. 3 also shows several types of heterogeneous multivariate temporal data, typical to patient measurement items, as follows: fixed frequency (FF) sampling, irregular sampling (IS), instantaneous events (IE), and raw STIs. The temporal abstraction process 106/206 (also in FIGS. 1 and 2, respectively) transforms raw time-stamped data points into a uniform representation of a series of STIs 120. The temporal abstraction may be performed in two ways: (1) a state abstraction (130 in FIG. 3), in which the values are classified into states based on a given threshold 132 and later concatenated into respective “H” or “L” STIS (at the right side of the figure); or (ii) gradient abstraction 108, in which measurement samples are segmented into increasing 134 or decreasing periods 136 according to their first derivative. For example, variables V1 are gradient abstracted by grouping them into increasing (Inc) and decreasing (Dec) STIs, while variables V2 are abstracted into STIs using state abstraction, based on a single threshold, into high (H) or low (L) states. Once a uniform collection of STIs is created, whether abstracted 120 or raw 122, a time interval analysis can be performed, such as TIRPS similarity, discovery of frequent TIRPS, or a continuous prediction of a TIRP's event of interest.

As discussed, the invention utilizes two simplifying techniques to provide an easy-to-handle (e.g., patient's) dataset: the abstraction 106/206, which converts the entire data into STIs, and the STIs combining 110/210 into TIRPs, given the temporal relations between the various STIs.

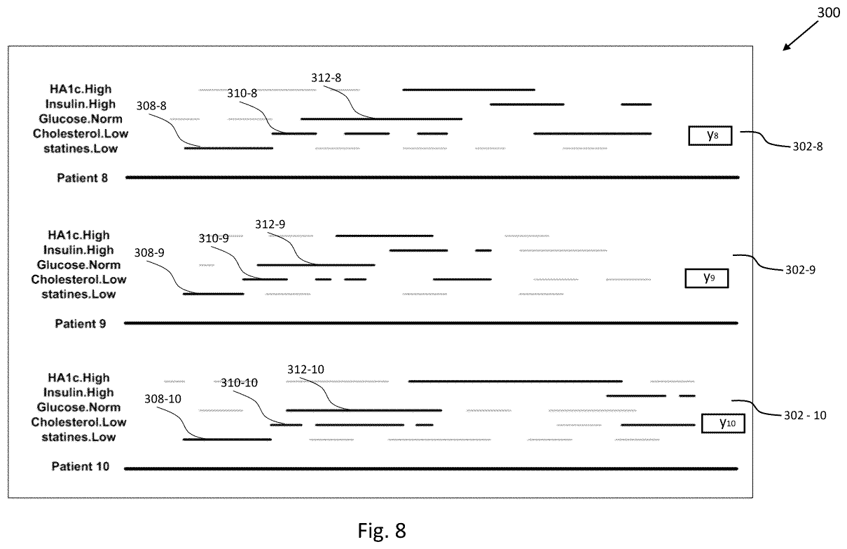

FIG. 8 shows an example 300 of measurement items, all reduced to a TIRPs form, as acquired from three patients, patient 8, patient 9, and patient 10. For each of the three patients, the collection of the black and gray TIRPs forms a patient dataset 302-8, 302-9, and 302-10 that ends in a specific event of interest y₈, y₉, y₁₀, respectively. The collection of black (dark) TIRPs for each patient represents a pattern that has been found, while all those gray TIRPs are irrelevant for the pattern. A careful inspection of the patterns in all the three patients shows a great similarity in their phases. For example, in all the three patients the pattern begins with a Statins. Low TIRP 308, continues with a Cholesterol. Low TIRP 310, which begins when the Statins. Low TIRP 308 ends, continues with a Glucose. Norm TIRP 312 that begins during TIRP 310, etc. An inspection of the rest of the three patterns shows additional TIRPs, all have the same phase relations within the pattern (while they may differ in their durations). The invention is based on finding in offline such common TIRPs patterns, all ending with the same event of interest. During real-time, and upon exposure of a partial pattern, a prefix calculation is periodically performed to determine the probability that this partial pattern will indeed end with the respective event of interest.

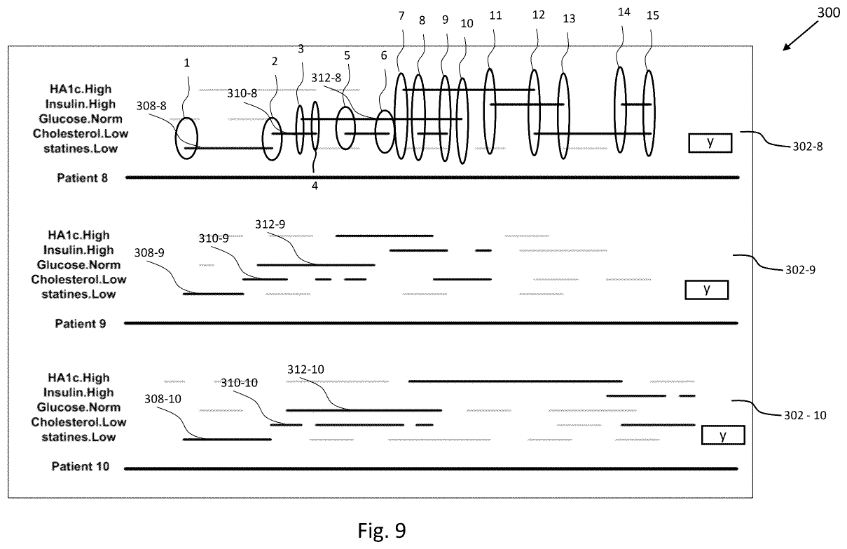

FIG. 9 shows all the “phase changes” (tieps) occurring within the pattern extracted from patient 8. In this case, the pattern includes 15 pattern changes, marked 1-15. The patterns that occurred in patients 9 and 10 include exactly the same number, 15, of phase changes, although these latter phase changes differ in their time and occurrence (and durations of their TIRPs items) relative to the pattern detected in patient 8. As discussed below, in one embodiment, the real-time verification may ignore the time of occurrence of the phase changes (and the TIRP durations) but will just consider the existence of the phase changes. In another embodiment, the time of occurrence of the phase changes (and the TIRP's STIs' durations) may also be considered, in addition to the existence of the phase changes.

Several definitions are used in the following description:

Definition 1—A symbolic time interval (STI) l=(s, e,sym), is a triplet of start time s ∈ [symbol] , end time e ∈ [symbol] , e≥s and a symbol sym (sym ∈ Σ)) from an ordered alphabet E.

Definition 2-A time interval endpoint (tiep) is a triplet (t, type, sym) consisting of a time stamp t ∈ [symbol] , an endpoint type, which can be either starting denoted by (+) or ending denoted by (−), and a symbol (sym ∈ Σ) from an ordered alphabet 2.

The total order of a set of tieps (see Definition 2) is defined based on their time stamps, which are real numbers. For an STI A=(Aₛ, Aₑ, “A”′), the starting and ending tieps are defined respectively as A+=(Aₛ, +, “A”) and A−=(Aₑ,−, “A”). Based on this notation and the total order between real numbers, the tieps can be used in inequalities defining temporal relations.

Definition 3 A TIRP Q is defined as a pair Q=(IS, R), where IS={I¹, . . . , I^{k}} is a series of k STIs, and R={r(Iⁱ, I^{j}): 1≤i<j≤k} is a set that defines all the temporal relations between each of the (k²−k)/2 pairs of STIs in IS.

Given an STI series of data, STIs-based patterns-TIRPs can be discovered utilizing TIRP mining techniques that use Allen's temporal relations. As noted in FIG. 4, Allen's temporal relations include seven temporal relations between a pair of STIs.

A TIRP is frequent if its vertical support exceeds a pre-defined minimum threshold. Given a database DB of |DB| unique entities (e.g., patients), the vertical support VS (DB, Q) of a TIRP Q is defined as the cardinality of the set DB^{Q} of distinct entities within which Q holds at least once, divided by |DB| (the total number of entities in DB), VS (DB, Q)=|DB^{Q}|/|DB|.

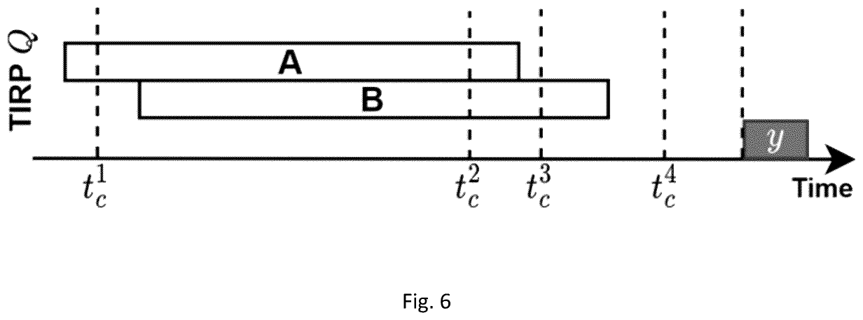

Reference is now made to FIG. 6. FIG. 6 illustrates how a TIRP Q's completion probability is estimated at any given time point

$$( e . g . , {t}_{c}^{2} ) .$$

Given a TIRP Q, which ends with an event of interest y, and a database DB (e.g., of patients data) a continuous prediction model m, which estimates the completion occurrence's probability of Q, is created. An estimation for the TIRP's completion occurrence (i.e., ending with y) is provided at each current time point to, and changes as a given instance of Q evolves over time. Let Pt denotes a prefix representing the observed part instance of Q at t_{c} (namely the part of Q preceding t_{c}) while Sₜ_{_{c}} denotes a suffix representing the remaining part instance of Q at t_{c} which is expected to occur. The process is interested in estimating Pr(Q|pₜ_{_{c}}), which represents the Q's completion probability, given an observed prefix pₜ_{_{c}} at time t_{c}:

$$Pr ( Q \vert {t}_{c} ) = Pr ( {s}_{{t}_{c}} \vert {p}_{{t}_{c}} ) = \frac{Pr ( {p}_{{t}_{c}} , {s}_{{t}_{c}} )}{Pr ( {p}_{{t}_{c}} )} = \frac{Pr ( Q )}{Pr ( {p}_{{t}_{c}} )} . \qquad ( 1 )$$

The calculation in Formula (1) answers the question: “Out of all the times we saw up to pₜ_{_{c′}}, how many times was it followed by sₜ_{_{c}} (i.e., Q has unfolded to completion)?” Thus, the number of times each pₜ_{_{c}} of Q occurs in the database and the number of times Pt is followed by sₜ_{_{c}} is counted during the offline procedure 100. Since the database DB comprises multiple entities, and each entity contains a lexicographically ordered STI series, instances of Q and pₜ_{_{c}} may be discovered more than once in a single entity. Probability calculations consider these instances as one occurrence.

Applying Formula (1) to a relatively simple example sheds light on the challenges that arise while continuously predicting a TIRP's completion (namely, predicting whether the TIRP will eventually end with Y). In FIG. 5, a TIRP Q={A overlaps B, A before y, B before y} is shown, together with four time-points

$${t}_{c}^{1} , {t}_{c}^{2} , {t}_{c}^{3} , and {t}_{c}^{4}$$

chosen to discuss the partial pattern's instance.

Calculating the numerator Pr(Q) is quite straightforward and is done by counting the number of times that A overlaps B is followed by A before y and B before y (i.e., Q). However, calculating the denominator Pr(pₜ_{_{c}}) is more challenging in some cases. At time points

$${t}_{c}^{1} or {t}_{c}^{3} ,$$

an STI that is not yet finished is involved, and pₜ_{_{c}} and sₜ_{_{c}} cannot be described with Allen's temporal relations. Instead, there is a need to use a different representation based on the STIs' tieps (Definition 2 above). For example, an STI A=(Aₛ, Aₑ, “A”), the starting and ending tieps are defined respectively as A+=(Aₛ, +, “A”) and A−=(Aₑ,−, “A”). The total order of a set of tieps (Definition 2) is defined based on their time stamps, which are real numbers. Thus, the tieps can be used in inequalities defining temporal relations, while their structure will be exploited in the following sections.

At

$${t}_{c}^{1} ,$$

the

$${p}_{{t}_{c}^{1}}$$

is “A that has started but not ended yet, “and, thus, Pr(A+) denotes the probability of seeing STI A that has started within the DB. In the learning stage (process 100 of FIG. 1a), and within the database DB, each STI has its starting and ending tieps, and thus, the probability Pr(A+) equals the probability of seeing STI A in the DB. Similarly, at

$${t}_{c}^{3} ,$$

the

$${p}_{{t}_{c}^{3}}$$

can be represented by the following inequality between the STIs' tieps: A+<B+<A−. However, in the learning stage, since STI B has started but not yet ended, its ending tiep B− has to satisfy

$${t}_{c}^{3}$$

<B− within database DB. Thus, the prefix's tiep ordering should be extended to

$${p}_{{t}_{c}^{3}} = A + < B + < A - < B -$$

to represent that STI B ended after B ended. The extended

$${p}_{{t}_{c}^{3}}$$

is equivalent to Allen's temporal relation A overlaps B (see FIG. 4).

Uncertainty is created at time point

$${t}_{c}^{2} .$$

### Since

$${p}_{{t}_{c}^{2}}$$

includes STIs A and B that have already started, but not yet ended

$$( i . e . , {t}_{c}^{2} < A - and {t}_{c}^{2} < B - ) ,$$

this results in uncertainty about which temporal relation between A and B will finally unfold. Three different temporal relations are possible: overlaps, contains, or finished-by, which should be considered and used in Formula 1.

Definition 4—An unfinished STI I* at time t_{c} is an STI whose starting tiep I*+satisfies 0≤I*≤t_{c} and whose ending tiep I*—satisfies t_{c}<I*−. The asterisk (*) indicates that an STI is unfinished throughout the text.

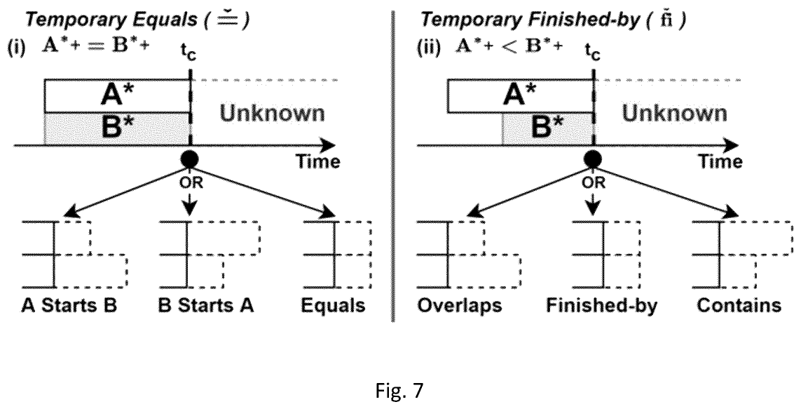

A pair of unfinished coinciding STIs A and B* may evolve into three possible temporal relations. The logic follows from the tieps representation of Allen's temporal relations that is presented in FIG. 4. FIG. 7(i) shows that in the case of the temporary equals (=̌) temporal relation, their temporal relation may eventually evolve into A starts B, B starts A, or stay A equals B.

The reason that A starts B and B starts A cannot be distinguished at t_{c} is that the exact temporal relation is determined by their end-times, which are not yet known. Similarly, the temporary finished-by (f̌i) temporal relation shown in FIG. 7(ii), may eventually evolve into three possible Allen's temporal relations: A overlaps B, A contains B, or stay A finished-by B.

As noted, TIRP can be represented by a series of starting and ending tieps instead of a series of STIs. To maintain the conjunction of pairwise temporal relations among the STIs, the set of tieps has to be transformed into a sorted tieps series, based on Allen's tieps representation (FIG. 4).

A TIRP is divided into TIRP-prefixes that are part of the TIRP's evolution process, which are created based on sub-sequences of the TIRP's tieps. In each TIRP-prefix, since the temporal relation between two unfinished STIs is uncertain, the temporary temporal relation ř is used to express the disjunction of possible temporal relations based on the unfinished coinciding STIs challenge logic explained in FIG. 6. For example, in FIG. 5, at

$${t}_{c}^{2} ,$$

it is known that Pt=A*++<B*+; thus, the TIRP-prefix is {A* f̌i B*), and the three following TIRPs may potentially evolve: {A overlaps B}, {A finished-by B}, or {A contains B}.

The inventors believe that there is no prior art that teaches the task of continuously aggregating predictions made using multiple TIRP completion models created in offline to estimate in real-time the probability of occurrence of an event of interest.

According to an embodiment of the invention, a continuous event prediction model M is created in offline given an event of interest y and an STI series database DB that comprises |DB| entities (e.g., patients), represented by STI series. During the real-time process 200, and during each current time point t_{c}, model M estimates the occurrence probability of the event of interest Pr(y|t_{c}) given the entity's data until t_{c}. Specifically, the inventors focused on the problem given DB and a set of models m₁, m₂, m₃ . . . all included within M, that estimate the completion of TIRPs that end with an event of interest. The following description begins in describing the models used for the completion of a single TIRP, and then continuous with the continuous aggregation of predictions made using multiple TIRP completion models to provide the event prediction model.

The predicted TIRP's completion probability changes only at time points where tieps appear. As discussed in detail when Formula 1 was explained, the probability of TIRP Q completion is Pr(Q)/Pr (pₜ_{_{c}}), where pₜ_{_{c}} is a TIRP-prefix of Q at time t_{c}.

Three alternative continuous prediction models (CPMs) to continuously estimate the probability of the TIRP completion based on the TIRP-prefixes, may be applied. The first, more simplistic, model is called the Segmented Continuous Prediction Model (SCPM). The two additional models, called the Fully Continuous Prediction Model (FCPM) and Machine Learning-Based Continuous Prediction Model (CPML), are more complex and incorporate the durations between the TIRP-prefix's consecutive tieps and thus provide estimated completion probability which is updated continuously (not in discrete times, as in the SCPM) as time progresses. References for the models can be found at:
(a) SCPM and FCPM: Itzhak, N., Jaroszewicz, S., & Moskovitch, R. (2023). Continuous prediction of a time intervals-related pattern's completion. Knowledge and Information Systems, 65 (11), 4797-4846;
(b) CPML: Itzhak, N., Jaroszewicz, S., & Moskovitch, R. (2023 May). Continuously predicting the completion of a time intervals related pattern. In Pacific-Asia Conference on Knowledge Discovery and Data Mining (pp. 239-251). Cham: Springer Nature Switzerland.

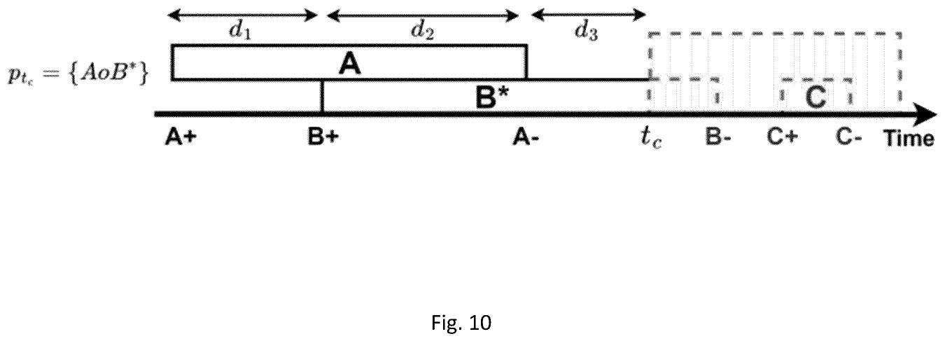

An example for a Machine Learning-Based Continuous Prediction Models (CPML) is now described. The durations between consecutive TIRP-prefixes tieps were used as features. Records for the classifier were created to represent the evolution of a TIRP over time. Multiple records were used as input for the classifier to consider all the time stamps for each evolving TIRP-prefix instance. The TIRP's duration elements that were not observed until time point t_{c} are set to zero. The recordation target for each instance was whether the instance was eventually unfolded to a TIRP's event of interest (completion). FIG. 10 shows an unveiled partial TIRP comprising elements A and B* used for predicting whether the TIRP will eventually complete, and possibly the occurrence time. In this example, time durations d1, d2, and d3 are represented by tieps A+, B+, A−, and to, that are used as features for the classifiers predicting the TIRP's completion probability and possibly occurrence time. Time durations d₁, d₂, and d₃ are based on tieps A+, B+, A−, and t_{c}, which are used as features for the classifiers for the TIRP'S completion prediction. For example, the TIRP-prefix instance {A overlaps B*} is represented by the time durations between the four consecutive tieps (ƒ₁=[A+, B+], ƒ₂=[B+, A−], ƒ₃=[A−, B− ], and ƒ₄=[B−, C+]), which are used as features ([ƒ₁, ƒ₂, ƒ₃, ƒ₄]) for the classifier. For the instance at to, the record values are ƒ₁=d₁, ƒ₂=d₂, ƒ₃=d₃, and ƒ₄=0.

The inventors used XGBoost (EXtreme Gradient Boosting) with Python 3.6, using the XGBoost package (xgboost. readthedocs.io) version 1.6.2. To find the optimal hyperparameters, the inventors performed a grid over search the learning rate η∈ {0.001,0.005,0.01,0.1}, number of estimators nₑₛₜᵢₘₐₜₒᵣₛ ∈ {25,50,100,250,1000}, and the maximum of number leaves in the trees Imax E {5,10,25,50,100,250}. Defaults were used for parameters not specified here. The fully continuous prediction model (FCPM) extends SCPM by also incorporating the distributions of durations of the TIRP-prefixes' consecutive tieps in order to better distinguish instances likely to evolve, or not, into the entire TIRP. To ensure clarity and prioritize space, SCPM and CPML were used. SCPM and CPML have comparable performance with FCPM, demand less explanation space, and are representative also of the FCPM approach.

Predictors for continuous aggregation of multiple TIRP completion are now discussed. An event prediction model M continuously aggregates prediction made by q independent models M={m₁, m₂, . . . , m_{q}}, where the model mᵢ (1≤i≤q) predicts the completion of a TIRP Qᵢ ∈ Φ={Q₁, Q₂, . . . , Q_{q}} ending with an event of interest y.

As shown, the use of multiple TIRPs is better than a model that consists of a single TIRP.

Since each TIRP Qᵢ may have multiple instances within the same entity, the inventors defined several terms that were used as part of the proposed models. Let

$${p}_{{t}_{c}}^{i . j}$$

denotes a prefix representing the j-th observed part instance of Qᵢ at t_{c}, while

$${s}_{{t}_{c}}^{i . j}$$

denotes a suffix representing the j-th remaining part instance of Qᵢ at t_{c} that is expected to occur. During the prediction stage, the event of interest occurrence probability is provided at each t_{c} and changes as a given entity evolves over time. An i-th model mᵢ ∈ M estimates the i-th TIRP Qᵢ ∈ Φ's completion for each of the vᵢ TIRP's instances

$${Υ}_{{t}_{c}}^{i} = \{ {p}_{{t}_{c}}^{i \text{.1}} , {p}_{{t}_{c}}^{i \text{.2}} , ... , {p}_{{t}_{c}}^{i . {v}_{i}} \}$$

that were detected in the entity's data until t_{c}. Overall, M at t_{c} provides the TIRPs' completion prediction for all detected TIRP instances

$${Υ}_{{t}_{c}} = {⋃}_{i = 1}^{q} {Υ}_{{t}_{c}}^{i} = \{ {p}_{{t}_{c}}^{i . j} : 1 \leq i \leq q , 1 < j \leq {v}_{i} \} .$$

A TIRP completion model, when applied to an instance γₜ_{_{c}}, serves as a predictor for M.

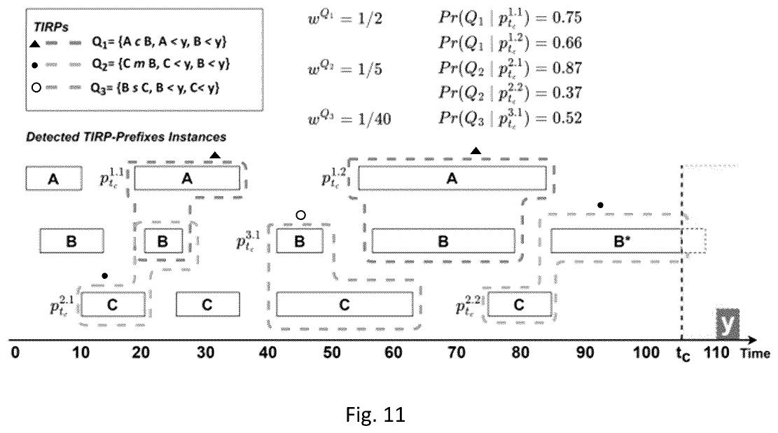

FIG. 11 provides an example of an entity (e.g., a patient) and its detected prefix instances of three TIRPs Φ={Q₁, Q₂, Q₃} that end with the event of interest y. The TIRPs describe an entity with three symbols A, B, and C until t_{c} to several attributes of multiple detected TIRP instances. In retrospect, it is known that this entity had an event of interest y in its data. The continuous event prediction model M={m₁, m₂, m₃} estimates at each t_{c} the y's occurrence probability, by continuously aggregating the Φ={Q₁, Q₂, Q₃}′ completion probabilities for the TIRP instances

$${Υ}_{{t}_{c}} = \{ {p}_{{t}_{c}}^{1.1} , {p}_{{t}_{c}}^{1.2} , {p}_{{t}_{c}}^{2.1} , {p}_{{t}_{c}}^{2.2} , {p}_{{t}_{c}}^{3.1} \} ,$$

detected until t_{c}. As shown in FIG. 11 with instances

$${p}_{{t}_{c}}^{1.1} and {p}_{{t}_{c}}^{1.2}$$

(colored blue—solid fill triangle), the instances of a TIRP can be detected multiple times in the same entity before the event of interest occurs. Additionally,

$${p}_{{t}_{c}}^{2.1} and {p}_{{t}_{c}}^{2.2}$$

are prefixes of TIRP Q₂ (colored yellow-solid fill circle) and have a different number of tieps, in which the earlier instance

$$( {p}_{{t}_{c}}^{2.1} )$$

is more advanced than the later one

$$( {p}_{{t}_{c}}^{2.2} ) .$$

The aggregation function is now described. Weighted aggregation functions give more weight to TIRPs that are believed to be more meaningful on the event prediction, and thus to their corresponding predictors, than other predictors in γₜ_{_{c}} using a weighted average with the weight vector W={w^{Qi}: 0<i≤q}:

$$Pr ( y \vert {t}_{c} ) = \frac{{\sum }_{i = 1}^{q} {\sum }_{j = 1}^{{v}_{i}} {w}^{{Q}_{i}} * Pr ( {Q}_{i} \vert {p}_{{t}_{c}}^{i . j} )}{{\sum }_{i = 1}^{q} {v}_{i} * {w}^{{Q}_{i}}} . \qquad ( 2 )$$

In Formula (2), in the numerator, a predictor for

$${p}_{{t}_{c}}^{i . j}$$

gets its

TIRP's weight w^{Qi}, and the denominator represents the summation of all predictors' weights for normalization. For example, in the upper right part of FIG. 11, the weight for each TIRP (e.g., w^{Q1}—the weight for the completion of Q₁) is presented in addition to the estimated TIRPs' completion probabilities. The weight vector W={w^{Q1}, w^{Qz}, w^{Q3}} defined in FIG. 11 assumes that more frequent TIRPs are more meaningful for the prediction. The weight vector W consists of weights that are computed for the TIRPS, based on the training data, where the weight of each TIRP is assigned to its detected prefixes' instances.

The following TIRP metrics were calculated based on the training set to estimate the meaningfulness of each TIRP: (1) the vertical support (VS) threshold represents the percentage of entities found to have at least one instance of the TIRP; (2) mean horizontal support (MHS) represents the averaged number of a TIRP'S instances that were detected in an entity; (3) mean duration (MD) refers to the TIRP's instances averaged durations from the instances' first start time till the last end time, averaged over all entities; (4) mean time from start (MTFS) refers to the TIRP's instances averaged times from the entity's data start time till the instances' first start time, averaged over all entities; and (5) TIRP size (Size) refers to the number of symbols in the TIRP.

Each aggregation function can be considered as giving the greatest weight to TIRPs' instances (i.e., predictors) with the greatest metric values (Greatest) or with its inverse version—the lowest weight to TIRPs' instances with the lowest metric values (Lowest). For example, the function TIRP size assumes patterns comprising more STIs are more meaningful (Greatest), while the inverse of TIRP size assumes that patterns that comprising fewer STIs are more meaningful (Lowest).

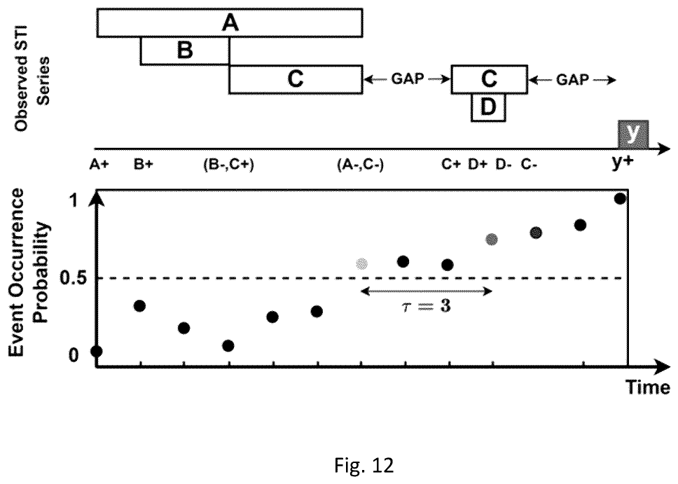

FIG. 12 shows an example where the event occurrence probability at any time point is based on the observed STI series. The prediction decision is made when the completion probability is higher than the threshold (the horizontal dashed line, 0.5 in this case), and a time delay t has been passed.

Early warning strategies may be used to decide that an event of interest will occur once there is a high likelihood, based on the event prediction model's estimated probabilities.

The following questions were verified by the inventors, as follows:
a. Is multiple TIRPs-based event prediction better compared to prediction based on a single TIRP?
b. Which continuous aggregation functions and whether using Greatest/Lowest performs best?
c. Which continuous prediction model (CPM), SCPM or CPML, performs best?

### Datasets

The inventors evaluated the proposed models using real-life medical and non-medical datasets: cardiac surgical patients (CSP), acute hypertensive episodes (AHE), diabetes (DBT), and elderly first injury fall (EFIF) datasets. The events of interest were defined, guided by relevant domain experts, as the first occurrence of the following: CSP-cardiac index lower than 2.5 L/min/m², AHE—the target onset, DBT-HbAIC greater than 9%, and EFIF-first fall in the elderly with a severe or moderate injury. Table 1 summarizes the main parameters of each dataset: entities (e.g., patients) number (#Ent), variables number (#Var), entities' maximum number of time stamps (#Timestamps), time granularity (Granularity), entities with the event of interest (#EntEvent), and where the values in the parentheses represent the percentage of #EntEvent out of #Ent.

TABLE 1
The evaluation datasets' parameters

| Name | #Ent | #Var | #Timestamps | Granularity | #EntEvent |
|---|---|---|---|---|---|
| CSP | 329 | 13 | 720 | minutes | 115 (36%) |
| AHE | 1,000 | 4 | 238 | hours | 500 (50%) |
| DBT | 1,710 | 12 | 24 | months | 239 (14%) |
| EFIF | 823 | 15 | 144 | weeks | 121 (15%) |

### Experimental Setup

The models were evaluated on the ability to predict an event of interest, using only time-based data. All the datasets were abstracted into STI series using EFD with three symbols per variable, as this configuration provided the best results. Then, the TIRPs were discovered from the STI data using the KarmaLego algorithm (described in R. Moskovitch and Y. Shahar, Fast time intervals mining using the transitivity of temporal relations, Knowledge and Information Systems, 42 (2015), pp. 21-48) and using Allen's seven temporal relations. A minimum vertical support of 40% was selected to balance the need for meaningful patterns within the entities (e.g., patients) experiencing the event, prioritizing their significance and potential impact. This choice also considered computational efficiency, as lowering the vertical support would drastically increase the number of patterns and require higher computational costs.

Only the discovered patterns that ended with the event of interest were used. Yet, all entities, with or without the event of interest, were used to learn the model's parameters and to evaluate the model. The inventors ran the experiments with ten-fold cross-validation, using target stratification. The target stratification was performed by maintaining the ratio between entities with and without the event of interest. This approach ensures a balanced representation of the target variable across different folds, allowing for a more reliable evaluation. Table 1 shows the percentage of entities containing the event of interest in each dataset.

### Evaluation Metrics

To evaluate the models' performance, a receiver operating characteristics (ROC) curve was calculated, and the corresponding area under the curve (AUROC). The decisions were made based on a prediction decision threshold, that was varied from 0 to 1, and the decision time delay t was varied using 0, 1, 2, 3, and 5 time units. The ROC was created by varying the prediction decision thresholds between 0 to 1 (FIG. 12).

### Experiments and Results

### Experiment 1: Single VS. Multiple TIRPs and Aggregation Functions:

In this experiment, to answer research questions (a) and (b), the inventors evaluated the use of the five novel continuous aggregation functions (VS, MHS, MD, MTFS, and TIRP size) of the multiple TIRPs' instances' completion predictors proposed above (with Greatest or Lowest weights) to determine which performs better. Additionally, the inventors evaluated whether learning a continuous model for event prediction based on multiple instances of several TIRPs (that all end with the event of interest) is better than a model based on multiple instances of a single TIRP. Each aggregation function is evaluated using a single TIRP with the greatest metric values for the event prediction while testing, separately, ten different TIRPs with the top greatest metric values and averaging their results. This approach is referred to as Single Top 10.

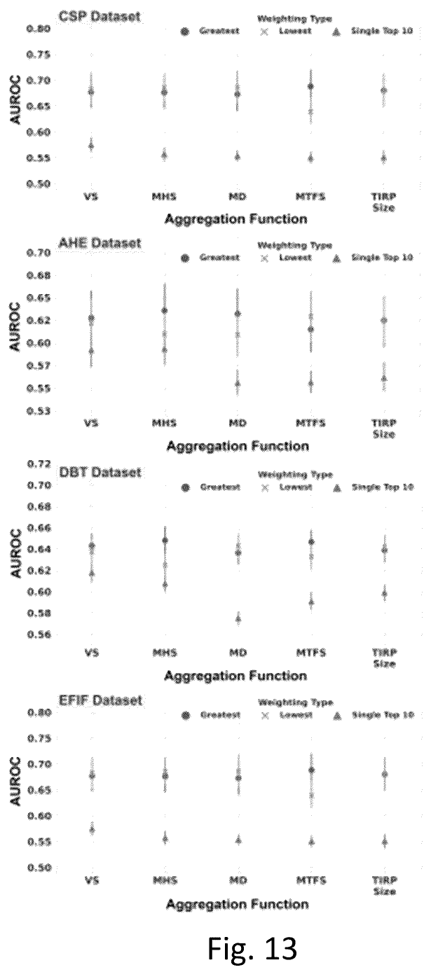

FIG. 13 presents the mean AUROC results for the weighted aggregation functions, averaging the results of the different t values. FIG. 13 also shows that overall, MTFS with Greatest performed best. Predictions based on instances of multiple different TIRPs perform better than instances of a single TIRP.

Single vs. Multiple TIRPs-answer to question (a): FIG. 13 shows that overall, using a model that is based on instances of multiple TIRPs having Greatest or Lowest weighting performed better than Single Top 10. For the AHE and DBT datasets, utilizing the Lowest weighting aggregation function for completion predictors of multiple TIRPS was better than Single Top 10, but not significantly.

Aggregation Functions-answer to question (b): FIG. 13 shows that for the CSP, DBT, and EFIF datasets, MTFS with Greatest and MD and TIRP size with Lowest resulted in the best performances. For the AHE dataset, MHS and MD with Greatest and MTFS with Lowest resulted in the best performances.

### Experiment 2: TIRPs-Based Prediction Parameters:

In this experiment, to answer question 3, the inventors evaluated the performance of SCPM and CPML. Four additional baseline models were evaluated for comparison with the proposed CPMs with multiple TIRPs and MTFS with Greatest as an aggregation function.

Continuous Prediction Model—answer to question 3:

The inventors tested the performance of SCPM, CPML, and the four baseline models that were applied on the raw heterogeneous multivariate temporal data: RawXGB (XGBoost using the raw data, not for TIRP's completion, as detailed above), ResNet, LSTM-FCN, and ROCKET. Reference for these models can be found at:
(a) XGBoost-Chen, T., & Guestrin, C. (2016). Xgboost: A scalable tree boosting system. In: Proceedings of the 22nd ACM SIGKDD International Conference on Knowledge Discovery and Data Mining, (pp. 785-794);
(b) ResNet-Wang, Z., Yan, W., & Oates, T. (2017). Time series classifcation from scratch with deep neural networks: A strong baseline. In: 2017 International Joint Conference on Neural Networks (IJCNN), (pp. 1578-1585). IEEE;
(c) LSTM-FCN-Karim, F., Majumdar, S., Darabi, H., & Chen, S. (2017). Lstm fully convolutional networks for time series classifcation. IEEE Access, 6, 1662-1669; and
(d) ROCKET-Dempster, A., Petitjean, F., & Webb, G. I. (2020). Rocket: exceptionally fast and accurate time series classifcation using random convolutional kernels. Data Mining and Knowledge Discovery, 34 (5), 1454-1495.

For the baseline models, the inventors applied linear interpolation on the raw data to solve the problem of the varying frequencies in the heterogeneous multivariate temporal data.

For all models, the inventors passed the raw data into the models using a sliding time window with the size of the entities' largest time duration in the training set. For RawXGB, an XGBoost model was implemented, in which the simple features (maximum, minimum, sum, median, mean, standard deviation, variance, and root mean square) were extracted from each time window of each temporal variable using tsfresh package (tsfresh. readthedocs.io). A grid search was applied over the same parameters and options described above.

ResNets, LSTM-FCN, and ROCKET, were also used as baseline models since they can be used to continuously estimate the probability of an event of interest occurring in heterogeneous multivariate temporal data. The ResNet and LSTM-FCN were trained with a maximum epoch of 2,000 and a batch size of 64, where the combinations of 500, 1,000, or 2,000 maximum epoch with batch sizes of 32, 64, 128, or 256 were tested.

The inventors used early stopping with a minimum change of 0.001 of the validation loss; lower than this value was not considered an improvement for the validation loss and was tolerated for 30 epochs. The model checkpoint procedure was modified to obtain the model that achieved the best prediction performance on the validation set. As increasing the number of kernels improved the results for ROCKET, 10,000 kernels were used, based on the maximal available resources. For parameters the inventors did not specify, the default values of the package were used.

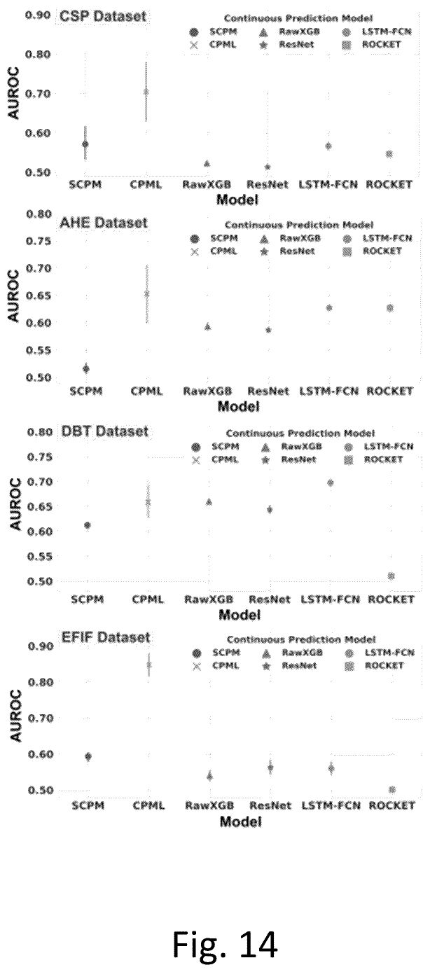

FIG. 14 shows that CPML performed significantly better than SCPM and much better than the other baseline models except for the DBT dataset. In contrast, LSTM-FCN results in a better performance than CPML on the DBT dataset, while RawXGB produces similar performances. Specifically, FIG. 14 presents the mean AUROC results for the event prediction models. Each point on the graph averages the results of the different t values. FIG. 14 also shows that for the CSP, AHE, and EFIF datasets, CPML performed significantly better than SCPM and the other baseline models. However, for the DBT dataset, LSTM-FCN results in a better result than CPML, while RawXGB achieved similar results to CPML. LSTM-FCN results in a better result than ResNet and ROCKET for the CSP and DBT datasets.

CPML resulted in wider confidence intervals than the other models, which can imply higher variability or outliers.

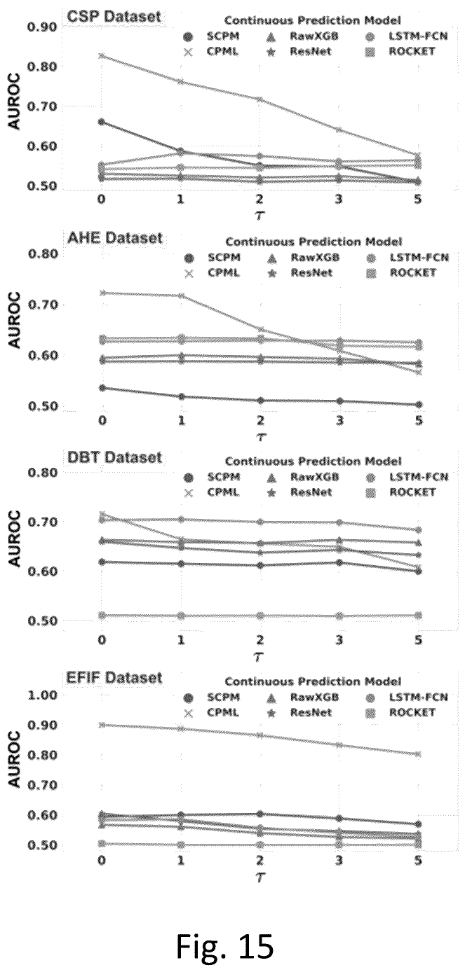

The t Parameter: The inventors evaluated the performances of the models while using the t parameter with 0, 1, 2, 3, or 5 time units. FIG. 15 presents the mean AUROC results for each model and T. FIG. 15 also shows that the t of zero time units performed best for CPML. However, the t parameter had a relatively small impact on the baseline models. In addition, LSTM-FCN performed better than the other baseline models.

To summarize, the inventors explored the concept of continuously predicting an event of interest based on a novel model that consists of multiple different TIRPs' unfolding instances. Also, the invention proposes novel weighted aggregation functions tailored for the combination of predictions of multiple TIRP completion models. This approach can be useful with STI series databases, but what makes it more interesting and more widely applicable in its use in heterogeneous multivariate longitudinal data, after employing temporal abstraction and transform of data into an STI series.

A rigorous evaluation was performed on four real-life datasets, demonstrating that overall the CPML performed better than the other models. The evaluation revealed that utilizing multiple TIRPs with suitable weighting strategies outperforms the use of a model consisting of a single TIRP with a top metric value. MTFS and MD showcased the highest AUROC as aggregation functions overall. Moreover, the comparison between SCPM and CPML demonstrated the superior performance of CPML in most cases, although LSTM-FCN and RawXGB exhibited competitive results for specific datasets.

The invention is particularly useful for predicting patients' events, such as recovery, complications, or death, and providing a respective boost or prevention measures at an early time. The evaluation by the process of the invention provides to the medical stuff indications that are in many cases hidden, or not simply observed given the vast amount of data that the physician should evaluate. The inventors evaluated the techniques of the invention for the prediction of the event of interest in entities' records (e.g., patients).

1. A method for real time predicting an event of interest of a specific patient, comprising:
an offline procedure comprising:
providing a retrospective patients' database;
defining an event of interest;
performing data abstraction on raw data of the retrospective database, resulting in a database of STIs (symbolic time intervals);
performing TIRP mining given a minimal vertical support on the database of STIs, resulting in a collection of frequent TIRPs (time intervals related patterns);
for the event of interest, selecting from the collection of frequent TIRPs those who end with the event of interest, or receiving from an external source one or more TIRPs that end with the event of interest;
based on said retrospective database, calculating a prediction model m for each TIRP ending with the event of interest, that indicates, for each prefix or a partial prefix of that TIRP, the probability of the prefix or partial prefix, if will appear in real time, to complete, and optionally also when, with the occurrence of said event of interest;
and,
a real-time TIRP-based event of interest prediction procedure comprising:
continuously abstracting at each given time newly received raw data relating to the specific patient, thereby creating an unfolding STI series;
for each given TIRP that ends with the event of interest, continuously detecting from the abstracted data a currently unfolding TIRP prefix;
given the currently unfolding prefix and a specific event of interest, referring to the model and receiving from the model m the respective probability of the unfolding prefix to eventually complete with the event of interest; and
continuously displaying to a user the updated probability of the currently unfolding prefix to complete with the event of interest.

2. The method of claim 1, wherein said one or more TIRPs are a plurality of TIRP instances, all ending with the same event of interest, and wherein a plurality of prediction models m, each relating to a different from said plurality of TIRP instances, are grouped within a global prediction model M.

3. The method of claim 2, wherein a plurality probabilities received, respectively for each TIRP instance, are aggregated before displaying a single aggregated probability to the user.

4. The method of claim 1, wherein the prefix probabilities within each said model m are listed only for time interval end points (tieps) of the respective TIRP

5. The method of claim 4, wherein the prefix probabilities within each model m are calculated by an SCPM (Segmented Continuous Prediction Model).

6. The method of claim 1, wherein the prefix probabilities within each model m are provided in a continuous time manner.

7. The method of claim 1, wherein the continuous time manner prefix probabilities within each said model m are calculated by either FCPM (Fully Continuous Prediction Model) or CPML (Machine Learning-Based Continuous Prediction Model).

8. The method of claim 1, wherein the calculation of each prefix probability within each model m is based on the rate in which same prefixes within the database of STIs have been eventually developed to a complete with the event of interest.

9. The method of claim 1, wherein said external source is a physician, a scientist, literature, or any framework built on large-language-model technology.

10. The method of claim 1, further displaying to the user one of: (a) when the event of interest is expected to occur; and (b) the instances on which the displayed probability is based.
